(19) United States
(12) Patent Application Publication
(10) Pub. No.: US 2009/0291987 A1
(43) Pub. Date: Nov. 26, 2009

(54) NOVEL POLYMORPH OF (5-AMINO-6-METHOXY-3-PYRIDINCARBONYL) CARBAMIC ACID 2-(S)-HYDROXYMUTILIN 14-ESTER

(75) Inventors: Steven Geoffrey Aitken, South Yorkshire (GB); Steven Dabbs, Essex (GB); Eric Hunt, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/721,465

(22) PCT Filed: Dec. 9, 2005
(86) PCT No.: PCT/EP05/13398

§ 371 Date: Jun. 12, 2007

Related U.S. Application Data
(60) Provisional application No. 60/635,663, filed on Dec. 13, 2004.

(51) Int. Cl. A61K 31/44; C07D 211/92

(52) U.S. Cl. 514/349; 546/285

(57) ABSTRACT

The present invention relates to a novel polymorph, to processes for the preparation of the polymorph and to the use of the polymorph in medicine, particularly antibacterial therapy.

### FIELD OF THE INVENTION

The present invention relates to a novel polymorph, to a process for the preparation of the polymorph and to the use of the polymorph in medicine, particularly antibacterial therapy.

### BACKGROUND OF THE INVENTION

International patent application WO 01/74788 describes the compound of formula (1)

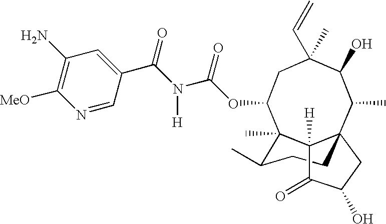

also known as (5-amino-6-methoxy-3-pyridinylcarbonyl)carbamic acid 2-(S)-hydroxymutilin 14-ester, as having antibacterial activity.

### SUMMARY OF THE INVENTION

It has now been found that (5-amino-6-methoxy-3-pyridinylcarbonyl)carbamic acid 2-(S)-hydroxymutilin 14-ester can be obtained in a novel polymorphic form, referred to herein as “Form A”, which may have one or more properties, for example bioavailability, which make it particularly useful as a pharmaceutical.

### BRIEF DESCRIPTION OF THE FIGURES

FIG. 1 is Fourier transform infra red (FTIR) spectrum of Form A.

FIG. 2 is an X-ray powder diffraction (XRPD) pattern of Form A.

FIG. 3 is a differential scanning calorimetry (DSC)/thermal gravimetric analysis (TGA) profile of Form A.

### DETAILED DESCRIPTION OF THE INVENTION

According to one aspect of the present invention there is provided a polymorphic form of (5-amino-6-methoxy-3-pyridinylcarbonyl)carbamic acid 2-(S)-hydroxymutilin 14-ester characterised in that it provides:

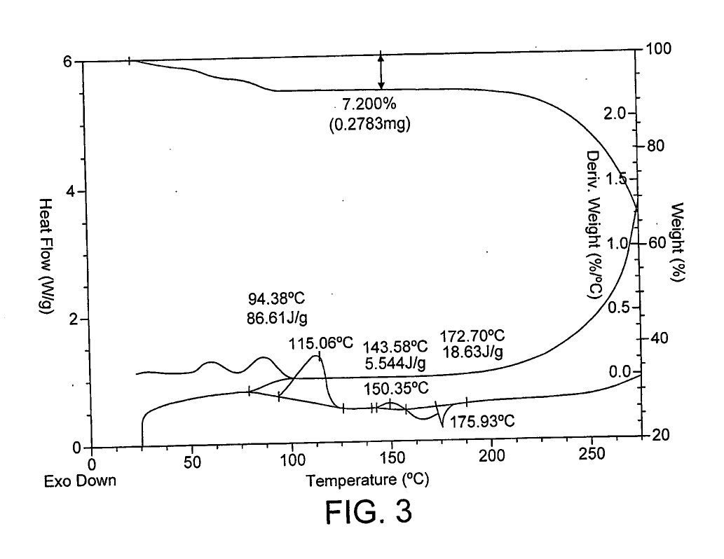

(i) a FTIR spectrum comprising peaks at about 914, about 1597 and about 1622 cm⁻¹; and/or
(ii) an XRPD pattern comprising peaks, expressed in degrees 2e at about 7.4, about 12.5 and about 21.9; and/or
(iii) a DSC profile substantially in accordance with FIG. 3: and/or
(iv) a TGA profile substantially in accordance with FIG. 3.

In one embodiment, the present invention provides Form A characterised in that it provides an FTIR spectrum comprising peaks substantially as set out in Table 1.

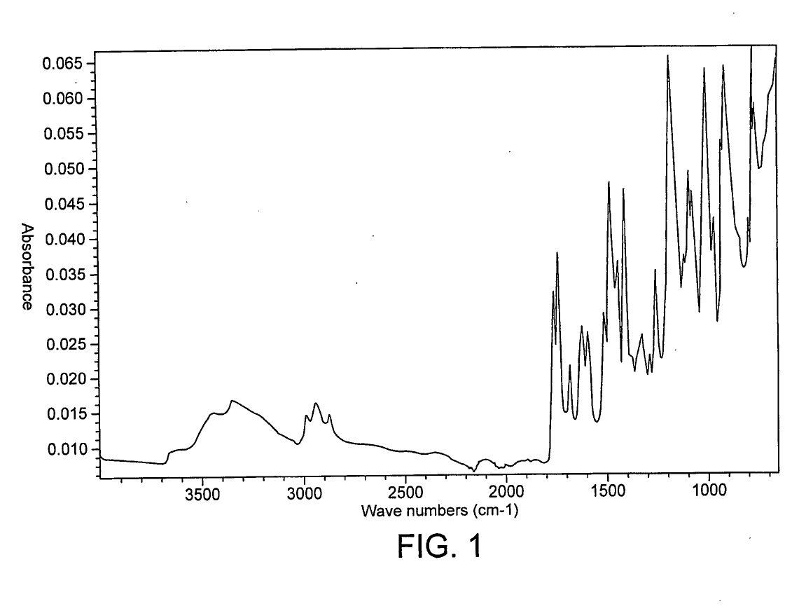

In another embodiment, the present invention provides Form A characterised in that it provides an FTIR spectrum substantially in accordance with FIG. 1.

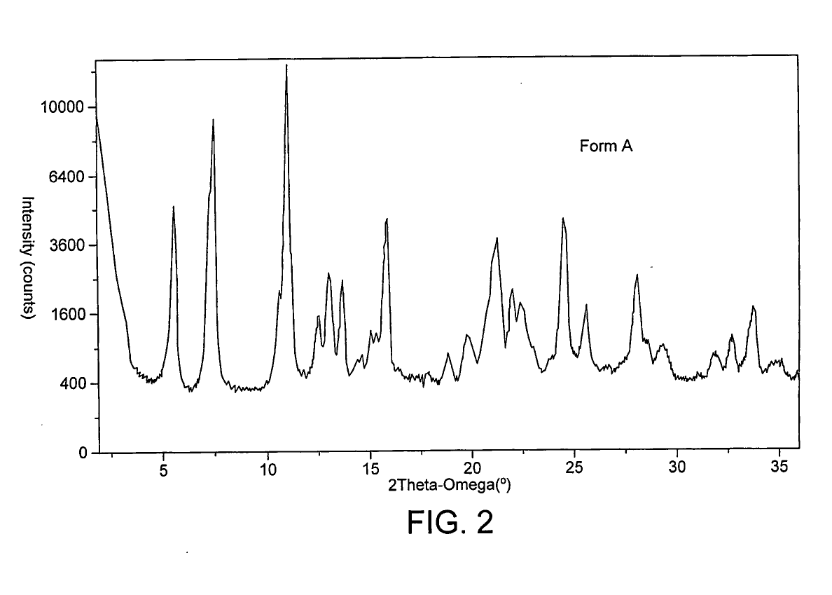

In another embodiment, the present invention provides Form A characterised in that it provides an XRPD pattern substantially in accordance with FIG. 2.

When it is indicated herein that there is a peak in a FTIR spectrum at a given value, it is typically meant that the peak is within ±2 cm⁻¹ of the value quoted.

When it is indicated herein that there is a peak in an XRPD pattern at a given value, it is typically meant that the peak is within ±0.1 of the value quoted.

The present invention encompasses Form A in pure form and Form A mixed with other materials, for example Form A mixed with alternative polymeric forms of (5-amino-6-methoxy-3-pyridinylcarbonyl)carbamic acid 2-(S)-hydroxymutilin 14-ester, amorphous (5-amino-6-methoxy-3-pyridinylcarbonyl)carbamic acid 2-(S)-hydroxymutilin 14-ester, or any other material.

In one embodiment, the present invention provides Form A in pure form.

In another embodiment, the present invention provides a mixture comprising more than about 60% Form A, for example more than about 80% Form A, such as more than about 90% Form A.

In another embodiment, the present invention provides Form A in crystalline form.

In a further embodiment, the present invention provides Form A in isolated form.

According to another aspect, the present invention provides a process for the preparation of Form A which comprises contacting an alternative polymorphic form of (5-amino-6-methoxy-3-pyridinylcarbonyl)carbamic acid 2-(S)hydroxymutilin 14-ester, referred to herein as “Form B”, with water. For example, Form B may be contacted with moist air such as air having a relative humidity of from about 25 to about 45%, for example about 30 to about 40%. The duration of contact is dependent on, for example, sample size. Suitably, Form B may be blown back with moist air for up to about 72 hours.

Form B may be prepared from (5-amino-6-methoxy-3-pyridinylcarbonyl)carbamic acid 2-(S)-hydroxymutilin 14-ester by crystallization from solvents such as methanol and hexane. For example, (5-amino-6-methoxy-3-pyridinylcarbonyl)carbamic acid 2-(S)-hydroxymutilin 14-ester in methanol may be heated until it dissolves, the mixture allowed to cool, and then hexane added. In one embodiment, (5-amino-6-methoxy-3-pyridinylcarbonyl)carbamic acid 2-(S)-hydroxymutilin 14-ester may be mixed with methanol, heated to an elevated temperature such as from about 30 to about 65° C. for up to about 2 hours and then gradually cooled to, for example, about 20° C. over up to about 1 hour, followed by addition of hexane and further cooling to, for example, about −10° C. If desired, Form B may be washed with a solvent such as methanol or hexane, for example hexane, before exposure to water to give Form A.

It is believed that Form A is a 1+x hydrate of (5-amino-6-methoxy-3-pyridinylcarbonyl)carbamic acid 2-(S)-hydroxymutilin 14-ester wherein the polymorph contains one equivalent of lattice water and one equivalent of water in a variable state. It is also believed that Form B is a dimethanol solvate of (5-amino-6-methoxy-3-pyridinylcarbonyl)carbamic acid 2-(S)-hydroxymutilin 14-ester.

(5-Amino-6-methoxy-3-pyridinylcarbonyl)carbamic acid 2-(S)-hydroxymutilin 14-ester may be prepared by any suitable method, for example by the procedure described in international patent application WO 01174788.

The polymorph of the present invention has antimicrobial properties and is therefore of use in therapy, in particular for treating microbial infections in animals, especially mammals, including humans, in particular humans and domesticated animals (including farm animals). The polymorph may be used for the treatment of infections caused by, for example, Gram-positive and Gram-negative bacteria and mycoplasmas, including, for example, Staphylococcus aureus, Staphylococcus epidermidis, Enterococcus faecalis, Streptococcus pyogenes, Streptococcus agalactiae, Streptococcus pneumoniae, Haemophilus sp., Neisseria sp., Legionella sp., Chlamydia sp., Moraxella catarrhalis, Mycoplasma pneumoniae or Mycoplasma gallisepticum. In one embodiment, the polymorph of the present invention may be used in the treatment of community acquired pneumonia (CAP), acute bacterial sinusitis (ABS) or skin and soft tissue infections (SSTI) including impetigo. For example, the polymorph of the present invention may be used in the treatment of community acquired pneumonia (CAP).

According to a further aspect, the present invention provides Form A for use in therapy.

The present invention also provides Form A for use in antimicrobial therapy.

The present invention also provides a method of treating microbial infections in animals, especially in humans and in domesticated mammals, which comprises administering Form A, or a composition according to the invention, to a patient in need thereof.

The invention further provides the use of Form A in the preparation of a medicament for use in the treatment of microbial infections.

It will be appreciated that reference to treatment includes acute treatment or prophylaxis as well as the alleviation of established symptoms.

Typically, a physician will determine the actual dosage which will be most suitable for an individual subject. The specific dose level and frequency of dosage for any particular individual may be varied and will depend upon a variety of factors including the activity of the specific polymorph employed, the metabolic stability and length of action of that polymorph, the age, body weight, general health, sex, diet, mode and time of administration, rate of excretion, drug combination, the severity of the particular condition, and the individual undergoing therapy.

The polymorph according to the invention may suitably be administered to the patient at a daily dosage of from 1.0 to 50 mg/kg of body weight. For an adult human (of approximately 70 kg body weight), from 50 to 3000 mg, for example about 1500 mg, of the polymorph according to the invention may be administered daily. Suitably, the dosage for adult humans is from 5 to 20 mg/kg per day. Higher or lower dosages may, however, be used in accordance with normal clinical practice.

While it is possible that, for use in therapy, the polymorph of the present invention may be administered as the raw chemical, typically the polymorph will be presented as a pharmaceutical formulation e.g. when the agent is in admixture with a suitable pharmaceutical excipient, diluent or carrier selected with regard to the intended route of administration and standard pharmaceutical practice.

More specifically, the polymorph and compositions according to the invention may be formulated for administration in any convenient way for use in human or veterinary medicine, by analogy with other antibiotics.

Accordingly, in one embodiment, the present invention provides a pharmaceutical composition or formulation comprising Form A in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another embodiment, the invention provides a pharmaceutical composition comprising Form A and a pharmaceutically acceptable excipient, diluent and/or carrier.

In another embodiment, the invention provides a pharmaceutical composition comprising, as active ingredient, Form A in association with a pharmaceutically acceptable excipient, diluent and/or carrier for use in therapy, and in particular, in the treatment of human or animal subjects suffering from a condition susceptible to amelioration by an antibacterial compound.

In another embodiment, the invention provides a pharmaceutical composition comprising a therapeutically effective amount of the Form A and a pharmaceutically acceptable excipient, diluent and/or carrier (including combinations thereof).

There is further provided by the present invention a process of preparing a pharmaceutical composition, which process comprises mixing Form A together with a pharmaceutically acceptable excipient, diluent and/or carrier.

The polymorph of the invention may be formulated for administration in any convenient way for use in human or veterinary medicine and the invention therefore includes within its scope pharmaceutical compositions comprising a polymorph of the invention adapted for use in human or veterinary medicine. Such compositions may be presented for use in a conventional manner with the aid of one or more suitable excipients, diluents and/or carriers. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s) or solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the polymorph of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e. g. as a carrier, diluent or solubiliser. Alpha-, beta- and gamma-cyclodextrins are most commonly used and suitable examples are described in WO 91/11172, WO 94/02518 and WO 98/55148.

The polymorph of the invention may be milled using known milling procedures such as wet milling to obtain a particle size appropriate for tablet formation and for other formulation types. Finely divided (nanoparticulate) preparations of the polymorph of the invention may be prepared by processes known in the art, for example see international patent application WO 02/00196 (SmithKline Beecham).

Routes for drug administration (delivery) include, but are not limited to, one or more of: oral (e. g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Typically, the polymorph of the invention will be administered orally.

If the composition comprises more than one active component, then those components may be administered by different routes.

There may be different composition/formulation requirements dependent on the different delivery systems. In one embodiment, the polymorph of the present invention is delivered orally. Hence, the polymorph is in a form that is suitable for oral delivery.

The polymorph according to the invention can be administered (e.g. orally) in the form of tablets, capsules, powders, granules, lozenges, creams, syrups, sprays, ovules, elixirs, or liquid preparations, for example solutions or suspensions, which may be formulated for oral use and which may contain flavouring or colouring agents, for immediate-, delayed-, modified-, sustained-, pulsed- or controlled-release applications.

Tablets and capsules for oral administration may be in unit dosage form, and may contain conventional excipients including, for example, granulation binding agents, for example, syrup, acacia, gelatin, sorbitol, tragacanth, polyvinylpyrrolidone, hydroxypropylmethylcellulose (HPMC), hydroxypropylcellulose (HPC) and sucrose; fillers, for example lactose, sugar, maize-starch, calcium phosphate, sorbitol or glycine; tabletting lubricants, for example magnesium stearate, stearic acid, glyceryl behenate and talc, polyethylene glycol or silica; disintegrants, for example starch (preferably corn, potato or tapioca starch), sodium starch glycollate, croscarmellose sodium or certain complex silicates; pharmaceutically acceptable wetting agents, for example sodium lauryl sulphate; and other excipients such as microcrystalline cellulose, sodium citrate, calcium carbonate, dibasic calcium phosphate and titanium dioxide. The tablets may be coated according to methods well known in normal pharmaceutical practice.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, including, for example, suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate or acacia; non-aqueous vehicles (which may include edible oils), for example almond oil, oily esters (for example glycerine), propylene glycol, or ethyl alcohol; preservatives, for example methyl or propyl p-hydroxybenzoate or sorbic acid; and, if desired, conventional flavouring and colour agents.

Where the polymorph is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

A polymorph or composition according to the invention is suitably administered to the patient in an antimicrobially effective amount.

A composition according to the invention may suitably contain from 0.001% by weight, preferably (for other than spray compositions) from 10 to 60% by weight, of the polymorph according to the invention (based on the total weight of the composition), depending on the method of administration.

When the compositions according to the invention are presented in unit dosage form, for instance as a tablet, each unit dose may suitably comprise from 25 to 1000 mg, for example from 50 to 500 mg, of the polymorph according to the invention.

The polymorph of the invention may also be used in combination with other therapeutic agents. The invention thus provides, in a further aspect, a combination comprising Form A together with a further therapeutic agent.

When the polymorph of the invention is used in combination with a second therapeutic agent active against the same disease state the dose of each compound may differ from that when the compound is used alone. Appropriate doses will be readily appreciated by those skilled in the art. It will be appreciated that the amount of a polymorph of the invention required for use in treatment will vary with the nature of the condition being treated and the age and the condition of the patient and will be ultimately at the discretion of the attendant physician or veterinarian. The polymorph of the invention may be used in combination with other antibacterial drugs such as a penicillin, a cephalosporin, a sulfonamide or an erythromycin.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the polymorph of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The polymorphs of the present invention may have one or more of the following advantageous properties such as improved: bioavailability, chemical/physical stability, and/or physical properties, for example solubility or flow properties etc.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

### EXAMPLE

### Preparation of Form A

A 15 L jacketed lab reactor was charged with (5-amino-6-methoxy-3-pyridinylcarbonyl)carbamic acid 2-(S)-hydroxymutilin 14-ester (2.650 kg) and methanol (11.26 L, 4.25 vol). The mixture was heated to until complete dissolution was observed. The solution was cooled slightly and charged with (5-amino-6-methoxy-3-pyridinylcarbonyl)carbamic acid 2-(S)-hydroxymutilin 14-ester seed. The suspension was held at above 50° C. for 30 minutes, then cooled to around 40° C. slowly. The suspension was stirred at around 40° C. for 30 minutes, then cooled to 20° C. over 45 minutes. Hexane (2.65 L, 1 vol) was charged to the suspension. The suspension was agitated at 20° C. for 50 minutes then cooled to −10° C. and held overnight. The product was isolated via filtration using Whatman 1 Qualitative filter paper. The filter cake (Form B) was washed with hexane (6.0 L, 2.3 vol) and blown back with air for 72 hr. The product, Form A, (2358 g) was discharged from the filter to pre-tared double-lined high density polyethylene bags in a cardboard cylinder.

### Characterising Data for Form A

### 1. Fourier Transform Infra Red (FTIR)

The FTIR spectrum was acquired on a Nicolet 550 Magna-IR equipped with a SensIR Durascope DATR (Diamond Attenuated Total Reflectance) accessory. A background was collected with no sample on the accessory. Then, approximately 2 mg of sample was placed on the diamond probe and flattened using a microscope slide. Pressure was applied to ensure the sample had good contact with the probe. Sixty-four co-added scans were collected at 4 cm⁻¹ resolution. The spectrum obtained is as shown in FIG. 1. Peak positions are listed in Table 1.

TABLE 1

| Selected vibrational bands for | Selected vibrational bands for |
|---|---|
| Form A in the region 600-1800 | Form A in the region 2800-3500 |
| cm − 1. values are in cm − 1. | cm − 1. Values are cm − 1. (B = broad) |
| 775.8 | 2870.1 (B) |
| 761.5 | 2938.0 |
| 798.5 | 2985.4 (B) |
| 913.5 | 3350.1 |
| 974.7 | 3443.7 |
| 1012.6 | |
| 1082.0 | |
| 1098.1 | |
| 1191.8 | |
| 1261.9 | |
| 1414.1 | |
| 1483.1 | |
| 1444.2 | |
| 1740.2 | |
| 1759.3 | |

### 2. X-Ray Powder Diffraction (XRPD)

XRPD data was acquired on a Philips X'Pert Pro Diffractometer. Approximately 30 mg of sample was gently flattened on a zero background silicon sample holder. The following acquisition parameters were used:

Scan range: 2-35 degrees two-theta
Generator power: 40kV, 40mA

### Radiation Source: Cu Ka

Step Time: 10.160 seconds
Step size: 0.0167 degrees two-theta per step

### Sample Rotation: 25 rpm

The XRPD pattern obtained is as shown in FIG. 2.

### 3. Thermal Data

### (a) Differential Scanning Calorimetry (DSC)

DSC data was acquired on a TA Q 1000 DSC system. The sample was placed into a pre-weighed aluminum DSC pan. The pan was sealed by applying pressure by hand and pushing the pan and the lid together (loose lid configuration). Data was acquired ramping at 10° C./min from ambient to 300° C.

### (b) Thermal Gravimetric Analysis (TGA)

TGA data was acquired on a TA Q500 TGA system. Sample is loaded onto a tared open Al pan. Data was acquired at a ramp at 10° C./min from ambient to 300° C.

The DSC and TGA data obtained are as shown in FIG. 3.
Alternatively Form A may be prepared on a smaller scale by taking a crude amorphous sample of (5-amino-6-methoxy-3-pyridylcarbonyl)carbamic acid 2-(S)-hydroxymutilin 14-ester (e.g. 44 g) and purifying it by chromatography using a biotage Flash 75 system eluting with a gradient of 75 to 100% ethyl acetate in 40-60 petroleum ether to give 38.5 g of amorphous white solid. This material can then be suspended in a mixture of methanol (166 ml) and water (10.7 ml) and warmed on a steam bath until complete dissolution is observed. The mixture should then be allowed to cool to approximately 45° C. followed by addition of (5-amino-6-methoxy-3-pyridylcarbonyl)carbamic acid 2-(S)-hydroxymutilin 14-ester seed crystals. The mixture should then be allowed to cool slowly for 4 hours and diethyl ether (80 ml) then added slowly. The product may be isolated by filtration and the solid washed with more diethyl ether (80 ml) followed by drying under high vacuum for 1 hour. The product may contained a trace of methanol by N.M.R analysis and can be kept in a dessicator under low vacuum for 60 hours over a dish of water to maintain a moist environment, followed by removal and drying under high vacuum for 1 hour. This drying process should be repeated for 24 hours. Finally the product should be stored open to the air for 5 days to give the product as Form A (22.89 g).

1. A polymorphic form of (5-amino-6-methoxy-3-pyridinylcarbonyl)carbamic acid 2-(S)-hydroxymutilin 14-ester characterised in that it provides:
(i) a FTIR spectrum comprising peaks at about 914, about 1597 and about 1622 cm⁻¹; and/or
(ii) an XRPD pattern comprising peaks, expressed in degrees 2θ at about 7.4, about 12.5 and about 21.9; and/or
(iii) a DSC profile substantially in accordance with FIG. 3; and/or
(iv) a TGA profile substantially in accordance with FIG. 3.

2. A polymorph according to claim 1 characterized in that it provides an FTIR spectrum comprising peaks substantially as set out in Table 1.

3. A polymorph according to claim 1 characterized in that it provides an FTIR spectrum substantially in accordance with FIG. 1.

4. A polymorph according to claim 1 characterized in that it provides an XRPD pattern substantially in accordance with FIG. 2.

5. A process for preparing a polymorph as claimed in claim 1 which comprises contacting Form B with water.

6. A pharmaceutical composition comprising a polymorph as claimed in claim 1 in association with a pharmaceutically acceptable excipient, diluent and/or carrier.

7-9. (canceled)

10. A method of treating microbial infections in animals, which comprises administering a polymorph as claimed in claim 1 to a patient in need thereof.
